(19) United States
(12) Patent Application Publication
(10) Pub. No.: US 2006/0200043 A1
(43) Pub. Date: Sep. 7, 2006

(54) Pap smear collection device with ejection sleeve

(76) Inventors: Joseph D. Jannetty, Naugatuck, CT (US); Richard Deslauriers, Woodbury, CT (US)

Correspondence Address: Joseph D. Jannetty, 62 Debbie Lane, Naugatuck, CT, 06770, US

(21) Appl. No.: 11/069,668

(22) Filed: Mar. 1, 2005

(51) Int. Cl. A61B 10/00

(52) U.S. Cl. 600/570

(57) ABSTRACT

Sample collection for the Pap smear sample is critical for accurate diagnosis. Improper sample collection, poor sampling, and/or cell preservation can render a Pap smear unsatisfactory for evaluation, requiring a repeat smear collection. If the Pap smear does not contain appropriate representative cells from the transformation zone and endocervical canal, the ability of the test to detect disease is very low. Likewise, if the preservation of the sample is compromised, the screener's ability to recognize abnormal cells is greatly diminished. It is generally understood that cervical samples should be harvested by a two-stage technique, which includes sampling of the endocervical canal with a cytobrush and obtaining a sample from the transformation zone with a spatula. The use of either the cytobrush or the spatula alone may be adequate but not as effective as the two-stage technique. Both the Cervex Brush and REG;(Unimar, Inc.) and the Accellon Combi & REG;(Medscand AB) are two collection devices which combine the action of the cytobrush and spatula, thus permitting broader sampling with a one stage technique. The standard method of transferring cervical cells from the collection device or complete transfer of the collection device into the liquid collection vial is often challenging. These challenges include but are not limited to: 1-spilling the sample, 2-dispersing the medium collection such that air born body secretions could contact unprotected health care workers, 3-missing the collection container and contaminating the sample. It is the goal of the current device embodiment to provide a simple and consistent method of transferring the entire collection specimen into the collection container to maximize cell collection while minimizing challenges of head disengagement. Such devices are not limited to the cervical cell cytology collection markets but extend to all cell collection methods were the entire sample is suspended in a liquid or similar medium. Such samples include oral cavity collections (throat swabs, vaginal cavity collections (STDs, pregnancy test), urethral cavity collections (male STDs) fornex collections (Alaph fetal protein), and rectal collections as well as open procedures requiring cell sampling.

### REFERENCES CITED [REFERENCED BY]

U.S. Patent Documents

| | | | |
|---|---|---|---|
| Re27915 | February, 1974 | Kohl | 128/757. |
| 3485236 | December, 1969 | Frost | 128/757. |
| 3613664 | October, 1971 | Willson | 128/756. |
| 3633565 | January, 1972 | McDonald | 128/2. |
| 3774590 | November, 1973 | McDonald | 128/2. |
| 3881464 | May, 1975 | Levene | 128/2. |
| 4078656 | March, 1978 | Crane et al. | 206/223. |
| 4127113 | November, 1978 | Nollan | 128/2. |
| 4384587 | May, 1983 | Milgrom | 128/757. |
| 4700713 | October, 1987 | Kist | 128/756. |
| 4754764 | July, 1988 | Bayne | 128/756. |
| 4759376 | July, 1988 | Stormby | 128/756. |
| 4762133 | August, 1988 | Bayne et al. | 128/756. |
| 4873992 | October, 1989 | Bayne | 128/756. |
| 4981143 | January, 1991 | Sakita et al. | 128/757. |
| 5022408 | June, 1991 | Mohajer | 128/756. |

Foreign Patent Documents

| | | | |
|---|---|---|---|
| 653880 | January, 1986 | CH | 128/757. |
| 2204496 | November, 1988 | GB | 128/757. |

### BACKGROUND

1. The Field of the Invention

The present invention relates generally to apparatus and methods for use in the collection of a Pap smear. More particularly, the present invention provides a novel design which permits the ejection of collection head into a storage or analysis container.

2. The Background of the Invention

Over the last 40 years, the death rate from cervical cancer has dropped dramatically due to the widespread use of the Papanicolaou (“Pap”) test. This test involves the collection of cells from the cervical face, the endocervical canal and occasionally from the vaginal wall.

For many years the standard method for collection of these cells has been a wooden or plastic spatula. A variety of slightly different shapes have been used in an effort to allow the clinician to collect cells from the cervical face as well as the endocervical canal. In some cases, the opposite end of the spatula was designed to collect a vaginal scraping.

Modifications in collection procedures have added a cotton swab to the technique in an effort to collect endocervical cells. The cotton swab is inserted into the cervical canal and rotated. Although the cotton swab may somewhat improve the yield, it is not abrasive enough to scrape the endocervical canal and consistently retrieve an adequate sample. Another modification made in an attempt to collect endocervical cells is what is called a “cytology brush.” This cytology brush has on the end of a long handle a conically-tapering brush made of spiraling bristles.

Once the collection procedure is completed the sample is transferred to either a microscopic examination slide or liquid medium for dilution. During this transfer it is often desirable or required to disengage the head from the handle assembly without contamination. Such efforts are either impossible or challenging and result in poorly controlled disengagement and risk spilling the specimen body fluids and exposing the practitioner to body fluids.

### BRIEF SUMMARY AND OBJECTS OF THE INVENTION

In view of the present state of the art, it is an object of the present invention to provide an apparatus and methods for the disengagement of various collection device configurations that control the transfer of said collection device to appropriate containers. It is another object of the present invention to provide apparatus and methods for disengaging collection device without contamination. It is yet another object of the present invention to provide apparatus and methods which reduce the potential for spilling the collection sample.

Additional objects and advantages of the invention will be set forth in the description which follows, and in part will be obvious from the description, or may be learned from the practice of the invention. The objects and advantages of the invention may be realized and obtained by means of the instruments and combinations particularly pointed out in the appended claims.

To achieve the foregoing objects, and in accordance with the invention as embodied and broadly described herein, the present invention is directed to an apparatus and methods of use which permit simultaneous contact and collection of cells from the ectocervix and endocervix. Accordingly, the device is provided with an elongated handle having a forward end and a rear end. The elongated handle having a ejection sleeve, thereby facilitating the controlled release of the collection device.

When the device is inserted into the uterus, the longitudinally extending member becomes inserted into the endocervical canal and the first sampling head makes contact with the endocervical walls. Simultaneously, the laterally extending member is pressed against the cervical face with sufficient forward pressure to release cells. The elongated handle being a flexible member can be curved so as to conform to the shape f the cervical face. Once the device is comfortably placed within the uterus, the device is then rotated 360 degrees several times. Endocervical cells and cells from the cervical face are collected onto the collection device.

Removing the collection device from the elongated handle may be accomplished by two methods. The first method is by utilizing the ejection sleeve to provide a forward force which results in the disengagement of the collection device. The second method involves holding the ejection sleeve stable and advancing the elongated handle.

### BRIEF DESCRIPTION OF THE DRAWINGS

In order that the manner in which the above-recited and other advantages and objects of the invention are obtained, a more particular description of the invention briefly described above will be rendered by reference to specific embodiments thereof which are illustrated in the appended drawings. Understanding that these drawings depict only typical embodiments of the invention and are therefore not to be considered limiting of its scope, the invention in its presently understood best mode will be described with additional specificity and detail through the use of the accompanying drawings in which:

FIG. 1 is a perspective view of one embodiment of the present invention.

FIG. 2 is a 3-dimensional view, illustrating the elongated handle.

FIG. 3 is a 3-dimensional view, illustrating the ejection sleeve.

FIG. 4 is a 3-dimensioal view, illustrating a cervical face collection device.

FIG. 5 is a 3-dimensional view, illustrating a endocervical canal collection device.

FIG. 6 is a 3-dimensioanl view, illustrating a sample collection device.

### DETAILED DESCRIPTION OF THE PREFERRED EMBODIMENTS

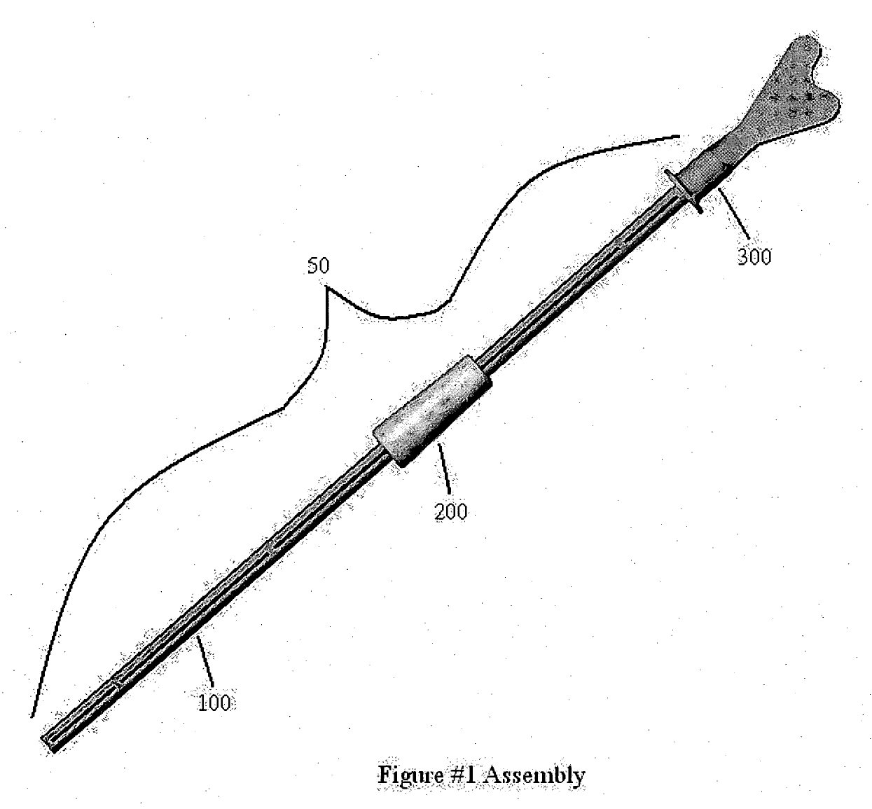

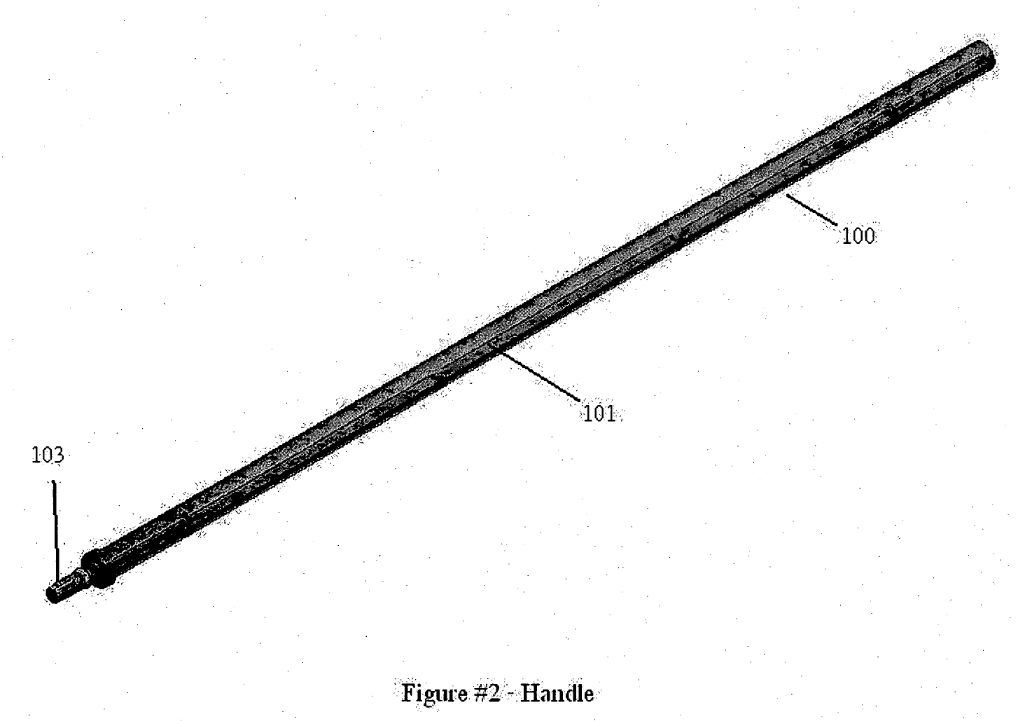

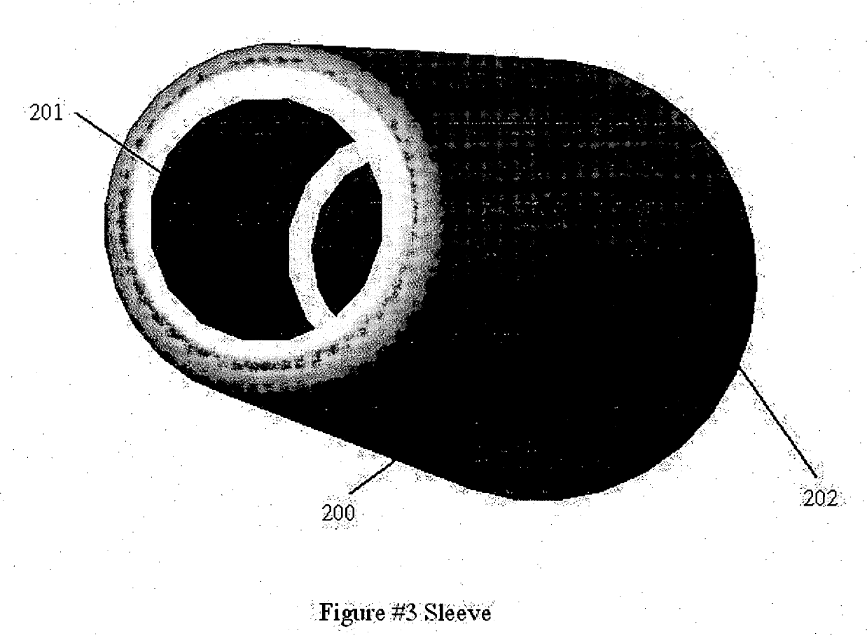

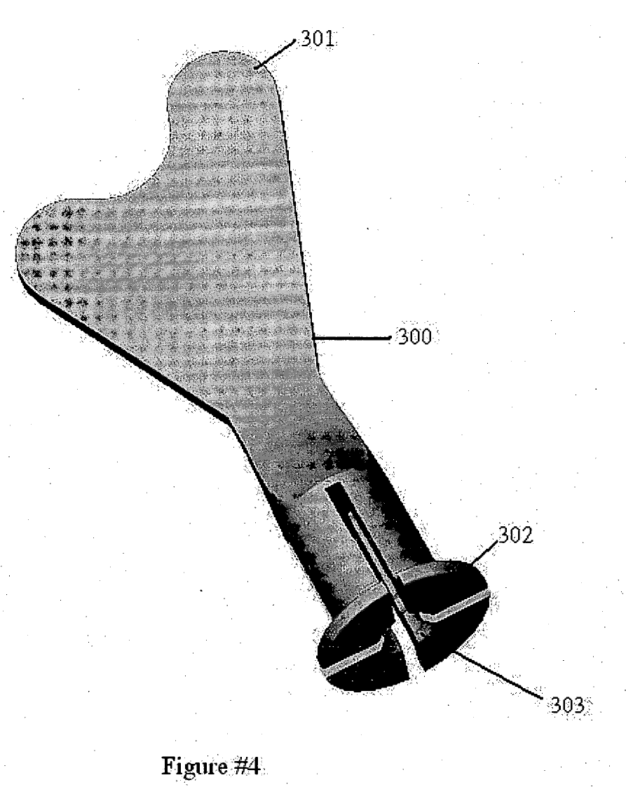

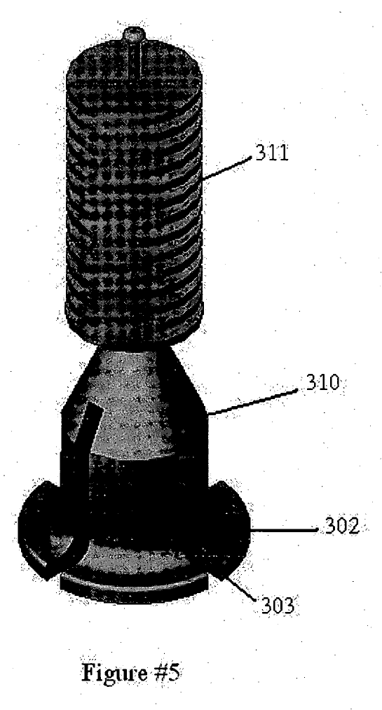

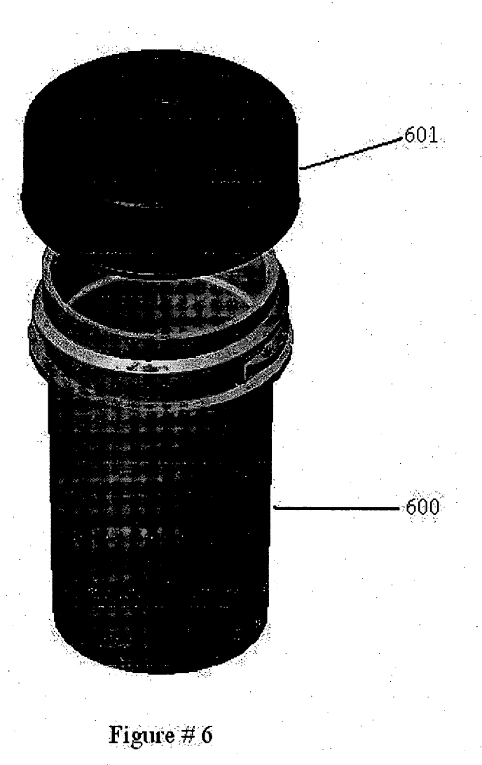

Reference is first made to FIGS. 1-6 which illustrate one presently preferred embodiment of the invention and its use. The device for simultaneously contacting and collecting cells from the endocervical canal and the cervical face of the uterus as 50 and can be seen in FIG. 1. The device 100 is comprised of an elongated handle and a external ridges 101 and distal interface 103. Although it can be appreciated that the handle 100 could be of a variety of lengths, the presently preferred length is about 7 inches. Handle 100 comprises longitudinally extending ridges 101 along the length of handle 100. These ridges 101 serve to provide better grip and ease of handling of the device 50. Handle 100 further comprises a forward end 103 adapted for engagement.

Ejection sleeve 200 comprises a distal end 201 and proximal end 202 adapted to encapsulate handle 100 and advances to interface with collection head 300 and or 310 at point 302 by providing linear force that expands interface 303 at distal interface 103.

As can be seen in FIG. 1, when the device 50 is inserted into the uterus the longitudinally extending member 100 becomes inserted into the endocervical canal contacting the collection site at distal end 301 and or 311.

In a presently preferred embodiment of the present invention, the extending member 50 can be slightly curved so as to conform comfortably to the cervical face, thereby permitting comfortable and full access. It can be appreciated that in older women whose cervices have expanded, embodiments with less of a curve may be preferred.

In a presently preferred embodiment of the present invention, sliding ejection sleeve 200 forward along 100 facilitates disengagement of collection device 311 and or 300 into collection container 600 once lid 601 is removed.

The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed and desired to be secured by United States Patent is:

1. A device for collection of tissue cells from a cervical face and endocervical canal of a uterus comprising:
(a) an elongated handle having a tissue sampling head and ejection sleeve;
(b) tissue sampling head, fixed to the distal end of said handle, collecting tissue cells from the cervical face and endocervical canal and disengaging from elongated handle.
(c) Ejection sleeve removing tissue sampling head from elongated handle.
The method of claim 1 wherein activation of ejection sleeve results in tissue sampling head reversibly disengaging from elongated handle.
The method of claim 1 wherein activation of ejection sleeve results in tissue sampling head irreversibly disengaging from elongated handle.
The method of claim 1 wherein activation of ejection sleeve is facilitated by holding the ejection sleeve stable and advancing the elongated handle, resulting in tissue sampling head reversibly disengaging from elongated handle.
The method of claim 1 wherein activation of ejection sleeve is facilitated by holding the ejection sleeve stable and advancing the elongated handle, resulting in tissue sampling head irreversibly disengaging from elongated handle.

2. A device for collection of tissue cells from a cervical face of a uterus comprising:
(a) an elongated handle having a tissue sampling head and ejection sleeve;
(b) tissue sampling head, fixed to the distal end of said handle, collecting tissue cells from the cervical face and disengaging from elongated handle.
(c) Ejection sleeve engaging tissue sampling head from elongated handle.
The method of claim 2 wherein activation of ejection sleeve results in tissue sampling head reversibly disengaging from elongated handle.
The method of claim 2 wherein activation of ejection sleeve results in tissue sampling head irreversibly disengaging from elongated handle.
The method of claim 2 wherein activation of ejection sleeve is facilitated by holding the ejection sleeve stable and advancing the elongated handle, resulting in tissue sampling head reversibly disengaging from elongated handle.
The method of claim 2 wherein activation of ejection sleeve is facilitated by holding the ejection sleeve stable and advancing the elongated handle, resulting in tissue sampling head irreversibly disengaging from elongated handle.

3. A device for collection of tissue cells from the endocervical canal of a uterus comprising:
(a) an elongated handle having a tissue sampling head and ejection sleeve;
(b) tissue sampling head, fixed to the distal end of said handle, collecting tissue cells from the endocervical canal and disengaging from elongated handle.
(c) Ejection sleeve engaging tissue sampling head.
The method of claim 3 wherein activation of ejection sleeve results in tissue sampling head reversibly disengaging from elongated handle.
The method of claim 3 wherein activation of ejection sleeve results in tissue sampling head irreversibly disengaging from elongated handle.
The method of claim 3 wherein activation of ejection sleeve is facilitated by holding the ejection sleeve stable and advancing the elongated handle, resulting in tissue sampling head reversibly disengaging from elongated handle.
The method of claim 3 wherein activation of ejection sleeve is facilitated by holding the ejection sleeve stable and advancing the elongated handle, resulting in tissue sampling head irreversibly disengaging from elongated handle.

4. A device for collection of tissue cells from a body cavity comprising:
(a) an elongated handle having a tissue sampling head and ejection sleeve;
(b) tissue sampling head, fixed to the distal end of said handle, collecting tissue cells from the body cavity and disengaging from elongated handle.
(c) Ejection sleeve engaging tissue sampling head.
The method of claim 4 wherein activation of ejection sleeve results in tissue sampling head reversibly disengaging from elongated handle.
The method of claim 4 wherein activation of ejection sleeve results in tissue sampling head irreversibly disengaging from elongated handle.
The method of claim 4 wherein activation of ejection sleeve is facilitated by holding the ejection sleeve stable and advancing the elongated handle, resulting in tissue sampling head reversibly disengaging from elongated handle.
The method of claim 4 wherein activation of ejection sleeve is facilitated by holding the ejection sleeve stable and advancing the elongated handle, resulting in tissue sampling head irreversibly disengaging from elongated handle.
